(19) United States
(12) Patent Application Publication
(10) Pub. No.: US 2025/0069220 A1
(43) Pub. Date: Feb. 27, 2025

(54) SYSTEMS AND METHODS FOR PARTITIONING MODELS OF ANATOMICAL STRUCTURES INTO FUNCTIONAL SEGMENTS

(71) Applicant: Intuitive Surgical Operations, Inc., Sunnyvale, CA (US)

(72) Inventors: Yuchen Xie, Cupertino, CA (US); Junning Li, San Jose, CA (US); Jin Young Hong, San Jose, CA (US)

(21) Appl. No.: 18/724,290

(22) PCT Filed: Dec. 30, 2022
(86) PCT No.: PCT/US2022/054389

§ 371 Date: Jun. 26, 2024

Related U.S. Application Data
(60) Provisional application No. 63/295,700, filed on Dec. 31, 2021.

(51) Int. Cl. G06T 7/00; G06T 5/30; G06T 5/70; G06T 7/11; G06T 7/62; G16H 30/40

(52) U.S. Cl. CPC G06T 7/0012; G06T 5/30; G06T 5/70; G06T 7/11; G06T 7/62; G16H 30/40; G06T 2207/20036; G06T 2207/20156; G06T 2207/30061; G06T 2207/30101

(57) ABSTRACT

An example method may include determining, by a computing system and based on a set of seeds determined from data representative of a labeled first tubular structure and a labeled second tubular structure in a model of at least a portion of an anatomical structure, a partitioning of the model into segments. The method may further include outputting, by the computing system, data representative of the segments.

### RELATED APPLICATIONS

The present application claims priority to U.S. Provisional Patent Application No. 63/295,700, filed Dec. 31, 2021, the contents of which is hereby incorporated by reference in its entirety.

### BACKGROUND INFORMATION

Medical procedures may include procedures on various organs of a patient (or any other subject). Certain organs of the patient may include functional segments that do not have visible boundaries. Determining boundaries of such functional segments may be useful. However, determining such boundaries may be nontrivial.

### SUMMARY

The following description presents a simplified summary of one or more aspects of the systems and methods described herein. This summary is not an extensive overview of all contemplated aspects and is intended to neither identify key or critical elements of all aspects nor delineate the scope of any or all aspects. Its sole purpose is to present one or more aspects of the systems and methods described herein as a prelude to the detailed description that is presented below.

An example method includes determining, based on a set of seeds determined from data representative of a labeled first tubular structure and a labeled second tubular structure in a model of at least a portion of an anatomical structure, a partitioning of the model into segments; and outputting data representative of the segments.

Another example method includes determining, based on a set of seeds determined from data representative of a labeled tubular structure in a model of at least a portion of an anatomical structure, a partitioning of the model into segments; and outputting data representative of the segments.

An example system includes a memory storing instructions and a processor communicatively coupled to the memory and configured to execute the instructions to determine, based on a set of seeds determined from data representative of a labeled tubular structure in a model of at least a portion of an anatomical structure, a partitioning of the model into segments; and output, data representative of the segments.

### BRIEF DESCRIPTION OF THE DRAWINGS

The accompanying drawings illustrate various embodiments and are a part of the specification. The illustrated embodiments are merely examples and do not limit the scope of the disclosure. Throughout the drawings, identical or similar reference numbers designate identical or similar elements.

FIG. 1 depicts an illustrative anatomical structure model partitioning system according to principles described herein.

FIGS. 2-3 depict illustrative configurations including anatomical structure model partitioning systems according to principles described herein.

FIGS. 4A-5C depict illustrative anatomical structure models according to principles described herein.

FIGS. 6-8 depict illustrative anatomical structure model partitions according to principles described herein.

FIG. 9 depicts an illustrative computer-assisted robotic medical system according to principles described herein.

FIGS. 10-12 depict illustrative methods of partitioning an anatomical structure model according to principles described herein.

FIG. 13 depicts an illustrative computing device according to principles described herein.

### DETAILED DESCRIPTION

Systems and methods for partitioning models of anatomical structures are described herein. An example method may include determining, by a computing system and based on a set of seeds determined from data representative of a labeled first tubular structure and a labeled second tubular structure in a model of at least a portion of an anatomical structure, a partitioning of the model into segments. The method may further include outputting, by the computing system, data representative of the segments.

Anatomical structures of a subject, such as organs, may include functional segments that may function independently from other segments. However, in some organs or other anatomical structures, such segments may not have visible boundaries. For instance, the lung may include bronchopulmonary segments that may individually be resected without affecting functions of other bronchopulmonary segments. A model of anatomical structure, such as an organ model, that shows such segments may be useful to a healthcare provider (e.g., a surgeon), such as by assisting the provider in determining optimal parameters of a medical procedure to minimize an effect (e.g., function loss) on the subject.

Systems and methods described herein may be configured to determine a partitioning of an anatomical structure model into segments, which may include such functional segments. Further, the system may output data representative of the segments. For instance, the system may output image data that represents the segments and/or boundaries of the segments (e.g., overlaid with the anatomical structure model). The system may output data representative of the segments to any system and/or component of a system that may use the data in any suitable manner, such as by using the data to facilitate the system and/or a user of the system ascertaining the segments.

Systems and methods described herein may provide various advantages and benefits. As described herein, for example, algorithms used by the system may result in partitioning of an anatomical structure model that more accurately reflects actual segments of the anatomical structure than conventional systems. Such accurate partitioning of anatomical structures may allow for better optimized and more efficient medical procedures that improve subject outcomes compared to conventional systems and methods.

Various illustrative embodiments will now be described in more detail. The illustrative embodiments describe partitioning of organ models for purposes of illustrations. The systems and methods may additionally or alternatively be applied to any suitable anatomical structures. The disclosed systems and methods may provide one or more of the benefits mentioned above and/or various additional and/or alternative benefits that will be made apparent herein.

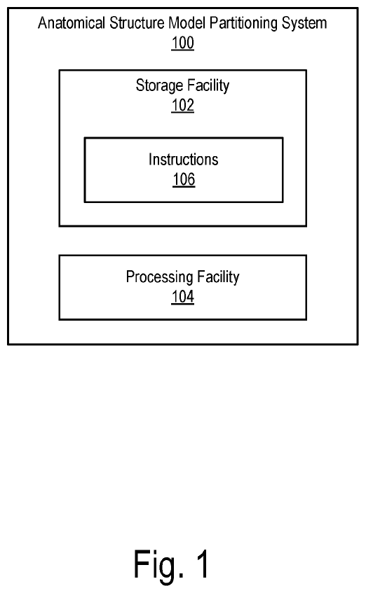

FIG. 1 illustrates an example anatomical structure model partitioning system 100 (“system 100”) configured to perform various operations described herein. As shown, system 100 may include, without limitation, a storage facility 102 and a processing facility 104 selectively and communicatively coupled to one another. Facilities 102 and 104 may each include or be implemented by hardware and/or software components (e.g., processors, memories, communication interfaces, instructions stored in memory for execution by the processors, etc.). For example, facilities 102 and/or 104 may be implemented by any component in a computer-assisted medical system configured to perform a medical procedure. As another example, facilities 102 and/or 104 may be implemented by a computing device separate from and communicatively coupled to a computer-assisted medical system. Although facilities 102 and 104 are shown to be separate facilities in FIG. 1, facilities 102 and 104 may be combined into fewer facilities, such as into a single facility, or divided into more facilities as may serve a particular implementation. In some examples, each of facilities 102 and 104 may be distributed between multiple devices and/or multiple locations as may serve a particular implementation.

Storage facility 102 may maintain (e.g., store) executable data used by processing facility 104 to perform one or more of the operations described herein. For example, storage facility 102 may store instructions 106 that may be executed by processing facility 104 to perform one or more of the operations described herein. Instructions 106 may be implemented by any suitable application, software, code, and/or other executable data instance. Storage facility 102 may also maintain any data received, generated, managed, used, and/or transmitted by processing facility 104.

Processing facility 104 may be configured to perform (e.g., execute instructions 106 stored in storage facility 102 to perform) various operations described herein. For example, processing facility 104 may be configured to determine, based on a set of seeds determined from data representative of a labeled vessel structure in a model, a partitioning of the model into segments and output data representative of the segments. These and other operations that may be performed by system 100 (e.g., processing facility 104) are described herein.

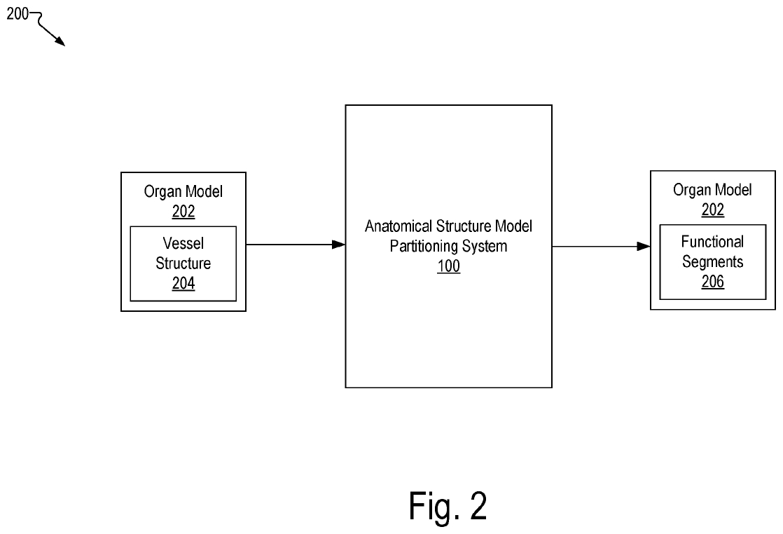

FIG. 2 illustrates an example configuration 200 of anatomical structure model partitioning system 100. Configuration 200 shows system 100 accessing (e.g., receiving, retrieving, generating, etc.) a model of an anatomical structure (e.g., an organ model 202). Organ model 202 may include any suitable model of at least a portion of any suitable organ of a subject. For instance, the model may include data representative of any suitable representation of the organ (or portion of the organ), such as data that represents the model in any suitable dimensions (e.g., in a three-dimensional coordinate system). The model may include visual and/or non-visual components. For example, the model may include any visual representation including two-dimensional imagery, three-dimensional imagery, four-dimensional imagery (three-dimensional imagery with a time component), color imagery, depth data, texture data, etc. The representation may further include non-visual representations, such as audio, tactile, etc. The representation may further include representations (visual and/or otherwise) of a function of the organ, which may or may not be otherwise visible (e.g., fluid flow within a vessel, bronchopulmonary segments of a lung, etc.). Data representative of organ model 202 may be in any suitable form that may be processed by one or more processors of a computing system.

As shown, organ model 202 may include a tubular structure (e.g., a vessel structure 204). Vessel structure 204 may be a structure of vessels (e.g., blood vessels, airway vessels, etc.) within the organ. The structure may represent various properties of the vessels, such as a pose (e.g., position, orientation, etc.) of the vessels, a geometry (e.g., a shape, size, etc.) of the vessels, a connectedness of the vessels, etc. Vessel structure 204 may be labeled to identify some or all of the vessels. For instance, vessels may be labeled by branch, such as branches of bronchi and bronchioles in a lung, branches of arteries in the lung, branches of portal veins and/or hepatic veins in the liver, etc. The labeling may be implemented in any suitable manner that distinguishes one label from another and/or that distinguishes labeled elements of organ model 202 from non-labeled elements of organ model 202. Examples of vessel structures of an organ model are further described herein. Further, while examples herein refer to organs, systems and methods described may be applied to any anatomical structure that may be resected. Such anatomical structures may include any suitable structure and/or group of structures and/or systems found in an anatomy, such as organs, neurological systems, etc. Organs may include any suitable group of tissues that includes a distinct structure and/or performs a distinct task, such as integumentary organs (e.g., skin, hair, nails, etc.), skeletal organs (e.g., bones, etc.), muscular organs (e.g., smooth muscles, cardiac muscles, skeletal muscles, etc.), circulatory organs (e.g., heart, arteries, veins, etc.), respiratory organs (lungs, diaphragm, larynx, etc.), digestive organs (e.g., stomach, intestines, liver, etc.), urinary organs (kidneys, ureters, bladder, etc.), immune system organs (e.g., lymph nodes, bone marrow, thymus, etc.), etc. Further, while examples herein refer to vessel structures, systems and methods described may utilize any tubular anatomical structures and/or branched anatomical structures, which may include vasculatures, airways, nerves, etc.

System 100 may receive data representative of organ model 202, which includes data representative of labeled vessel structure 204. System 100 may determine a set of seeds based on vessel structure 204. Each seed may include any point (e.g., pixel, voxel, etc.) or set of points (e.g., points defining one or more objects or structures or portions of objects or structures, a set of points derived from one or more objects or structures, an arbitrary set of points, etc.) that may be used for partitioning. Based on the set of seeds, system 100 may determine a partitioning of organ model 202 that defines segments of organ model 202. The segments may include functional segments 206 of the organ. A functional segment may include any portion of the organ that has at least some independent functionality from other segments of the organ. For instance, the organ may be a lung and a functional segment may be a bronchopulmonary segment, which may be resected without affecting functions of neighboring bronchopulmonary segments. Other examples may include functional segments of a liver, a foreign growth (e.g., a tumor) in an organ, etc.

System 100 may output data representative of functional segments 206. The data may include any suitable representation of the segments. For instance, system 100 may output organ model 202 with functional segments 206 such that a display of output organ model 202 may include a visual representation of the organ with labeled functional segments 206. For example, each segment may be represented in a different color or other visually distinguishing manner. Additionally or alternatively, boundaries of each segment may be displayed on organ model 202 in any suitable manner. Additionally or alternatively, system 100 may output data representative of functional segments 206 to any other suitable system and/or component of a system for further processing. Examples of outputs of partitioned organ models are further described herein.

Output of system 100 may be used in various ways. For example, such output may be applied for optimizing parameters for a medical procedure and/or a medical session. A medical procedure may include any activity conducted on a patient, such as minimally-invasive surgical procedures, open surgical procedures, non-surgical procedures, diagnostic procedures, therapeutic procedures, procedures in clinical, non-clinical, and/or training settings, etc. A medical session may include any activities associated with preparing for, performing, and finalizing the medical procedure, such as pre-procedure activities, intra-procedure activities, and/or post-procedure activities. Additionally or alternatively, output of system 100 may be applied for activities associated with a medical session, such as planning the medical procedure, evaluating the medical procedure, etc.

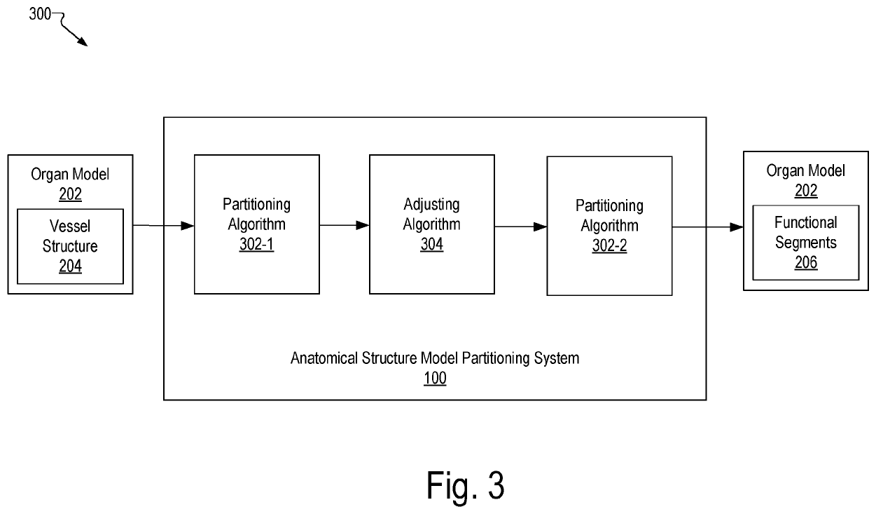

FIG. 3 illustrates another example configuration 300 of anatomical structure model partitioning system 100. As shown in configuration 300, system 100 includes partitioning algorithms 302 (e.g., partitioning algorithm 302-1 and 302-2) and an adjusting algorithm 304.

System 100 may access organ model 202, which may include vessel structure 204. In some examples, organ model 202 may include a plurality of different tubular structures, based on which a set of seeds may be determined for partitioning organ model 202. For example, organ model 202 may be a model of a lung of a subject (e.g., a binary mask of a lobe of the lung) and a first tubular structure may include an artery structure and a second tubular structure may include an airway (e.g., bronchi and bronchioles) structure. In some examples, each of these tubular structures may be labeled by branch, for instance, to at least a fourth generation of branch division. The labeled vessel structure may be provided as a label map to system 100.

System 100 may determine a set of seeds based on vessel structure 204. Each seed of the set of seeds may be based on each labeled portion of vessel structure 204. If organ model 202 includes more than one vessel structure 204, each seed may be based on any suitable combination of labeled portions of the plurality of vessel structures. For instance, for the lung organ model including an artery vessel structure and an airway vessel structure, each seed may include a respective airway branch and a corresponding closest artery branch. Alternatively, the seed may include structures derived from the airway and/or artery branch, such as a weighted combination of points of respective airway and corresponding closest artery branches, a central path of one or both vessel structures, etc.

The seeds may be used for partitioning organ model 202, such as by partitioning algorithm 302-1. Partitioning algorithm 302-1 may include any suitable space partitioning algorithm, such as a nearest neighbor algorithm. For instance, partitioning algorithm 302-1 may apply a nearest neighbor algorithm to determine a Voronoi partitioning of organ model 202 based on the set of seeds. Thus, each point of organ model 202 may be assigned to the label of its nearest seed (e.g., artery and/or airway branch). Based on partitioning algorithm 302-1, system 100 may determine a first partitioning of organ model 202 based on the set of seeds.

System 100 may adjust the first partitioning, such as by applying adjusting algorithm 304. Adjusting algorithm 304 may include any suitable algorithms that may adjust the first partitioning in any suitable manner. For instance, adjusting algorithm 304 may include one or more of a smoothing algorithm, a denoising algorithm, a tolerance algorithm, a dilation algorithm, an expansion algorithm, or any other such algorithm that may adjust the first partitioning.

For example, adjusting algorithm 304 may include a smoothing algorithm (e.g., a morphological erosion algorithm) that is applied to the first partitioning, followed by an expansion algorithm (e.g., a morphological dilation algorithm). For instance, the first partitioning may be morphologically eroded with a ball structuring element with a radius of 10 millimeters (mm), and then morphologically dilated with a ball structuring element with a radius of 7 mm. (Other suitable sizes of ball structuring elements or other suitable structuring elements of suitable sizes may be used in other implementations.) As a result of morphologically eroding and dilating the first partitioning, the first partitioning may be adjusted to determine a set of augmented seeds. Each augmented seed may include its respective initial seed of airway and artery branch, which may occur as a result of application of the adjusting algorithm or by merging a result of the adjusting algorithm with the initial seed.

Such adjusting may result in augmented seeds that more closely resemble a shape that corresponds to expected segment shapes of the organ than the initial set of seeds. For instance, the first partitioning may result in partitions that are more vessel or tube shaped than would be expected of bronchopulmonary segments because of the shape of the initial set of seeds. By adjusting the seeds using a morphological erosion and a morphological dilation (or any suitable adjusting algorithm), the augmented set of seeds may be closer to rounded shapes that encompass each respective airway and corresponding artery branch.

Based on these augmented seeds, system 100 may apply partitioning algorithm 302-2. Partitioning algorithm 302-2 may be any suitable partitioning algorithm, which may be a same or different algorithm as partitioning algorithm 302-1. For example, partitioning algorithm 302-2 may be a same partitioning algorithm as partitioning algorithm 302-1, applying a nearest neighbor algorithm to determine a second Voronoi partitioning of organ model 202 based on the augmented seeds. The second partitioning may define boundaries of the segments, which may be output by system 100 as functional segments 206 (e.g., a label map partitioning the lobe into functional segments 206). The second Voronoi partitioning based on augmented seeds may provide for each point of organ model 202 being assigned a segment label, as would be expected of actual bronchopulmonary segments.

While configuration 300 shows these particular algorithms in this particular order, other configurations may include additional algorithms, fewer algorithms, or a different order of algorithms. For instance, in some examples, system 100 may omit partitioning algorithm 302-2 and output segments based on an adjusting of the first partitioning generated by partitioning algorithm 302-1. In other examples, system 100 may include another adjusting algorithm after partitioning algorithm 302-2 that further adjusts the second partitioning in any suitable manner.

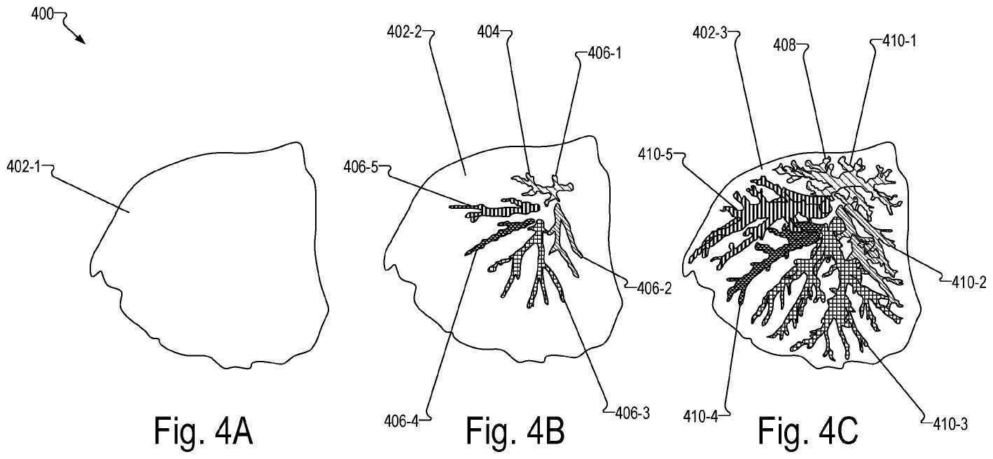

FIGS. 4A-4C illustrate an example configuration 400 of an organ model 202. FIG. 4A includes an image 402-1 of organ model 202, which shows a lobe of a lung of a subject. In this example, image 402-1 may be an image showing one perspective (e.g., an anterior view) of a 3D model of the lobe.

FIG. 4B includes an image 402-2 of organ model 202, which shows an airway vessel structure 404. Airway vessel structure 404 may be labeled based on branches of the vessel structure, such as airway vessel branches 406 (e.g., airway vessel branches 406-1 through 406-5). Each airway vessel branch 406 may be depicted in a manner different from each other, such as in a different color or any other suitable differentiated representation.

FIG. 4C includes an image 402-3 of organ model 202, which shows an artery vessel structure 408. Artery vessel structure 408 may be labeled based on branches of the vessel structure, such as artery vessel branches 410 (e.g., artery vessel branches 410-1 through 410-5).

Each artery vessel branch 410 may correspond to a respective airway vessel branch 406. The correspondence of artery vessel branches 410 with airway vessel branches 406 may be based on proximity, on a predetermined labeling, or any other suitable manner. Corresponding artery vessel branches 410 and airway vessel branches 406 may be represented with a same differentiation (e.g., a same color, etc.).

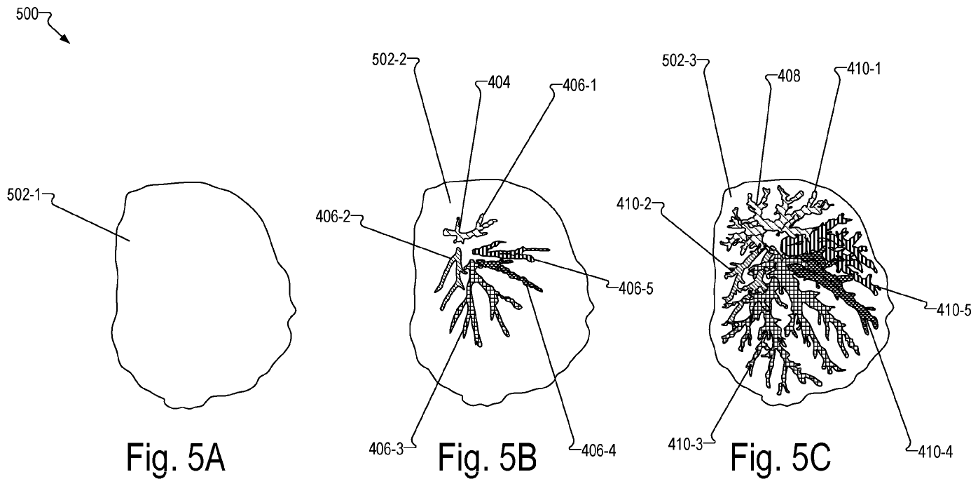

FIGS. 5A-5C illustrate another example configuration 500 of organ model 202, which may show another perspective (e.g., a posterior view) of organ model 202 of configuration 400. As such, FIG. 5A shows an image 502-1 of organ model 202 that shows the posterior view of the lobe of the lung of the subject shown in image 402-1. FIG. 5B includes an image 502-2 that shows organ model 202 and airway vessel structure 404 with airway vessel branches 406 from the posterior view. FIG. 5C includes an image 502-3 that shows organ model 202 and artery vessel structure 408 with artery vessel branches 410 from the posterior view.

As described, a set of seeds may be determined based on airway vessel structure 404 and artery vessel structure 408. For instance, airway vessel branch 406-1 and artery vessel branch 410-1 may constitute a first seed (e.g., each voxel labeled on either branch may be included as the first seed). Similarly, airway vessel branch 406-2 and artery vessel branch 410-2 may constitute a second seed, and so forth to determine five seeds based on airway vessel branches 406 and artery vessel branches 410. Based on such a set of seeds, system 100 may determine a partitioning of organ model 202 into segments and output data representative of the segments.

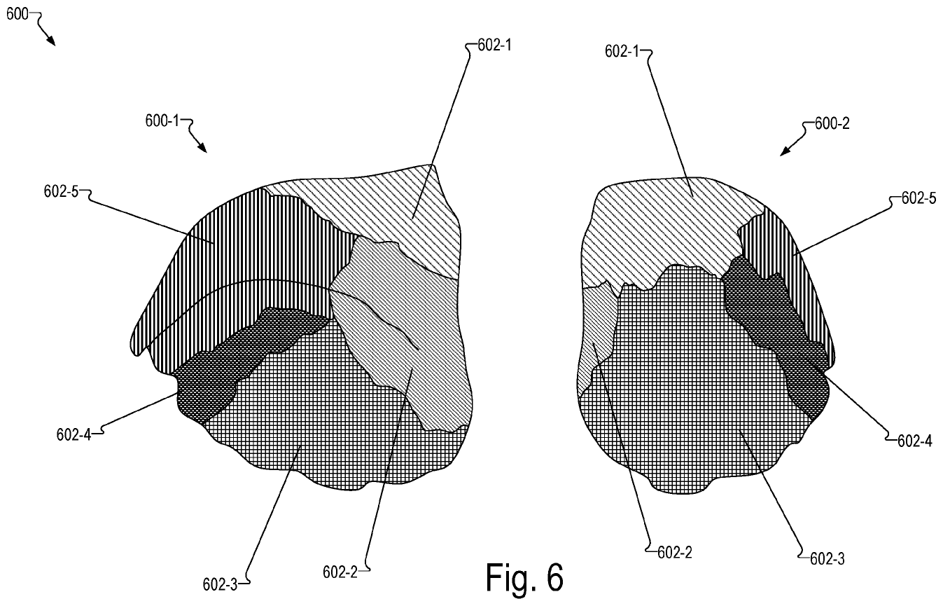

For instance, FIG. 6 illustrates example images 600 (e.g., images 600-1 and 600-2) of data output representative of segments determined based on the set of seeds as described in configurations 400 and 500. Image 600-1 shows an anterior view of organ model 202 partitioned into segments 602 (e.g., segments 602-1 through 602-5), while image 600-2 shows a posterior view of organ model 202 partitioned into segments 602.

Segments 602 may be determined based on the seeds in any suitable manner as described herein. As shown, segments 602 may be labeled based on seeds determined based on vessel structure 204. In this example, each seed includes a respective airway vessel branch 406 and corresponding artery vessel branch 410. Thus, each segment 602 is shown with a same differentiating representation as its respective seed.

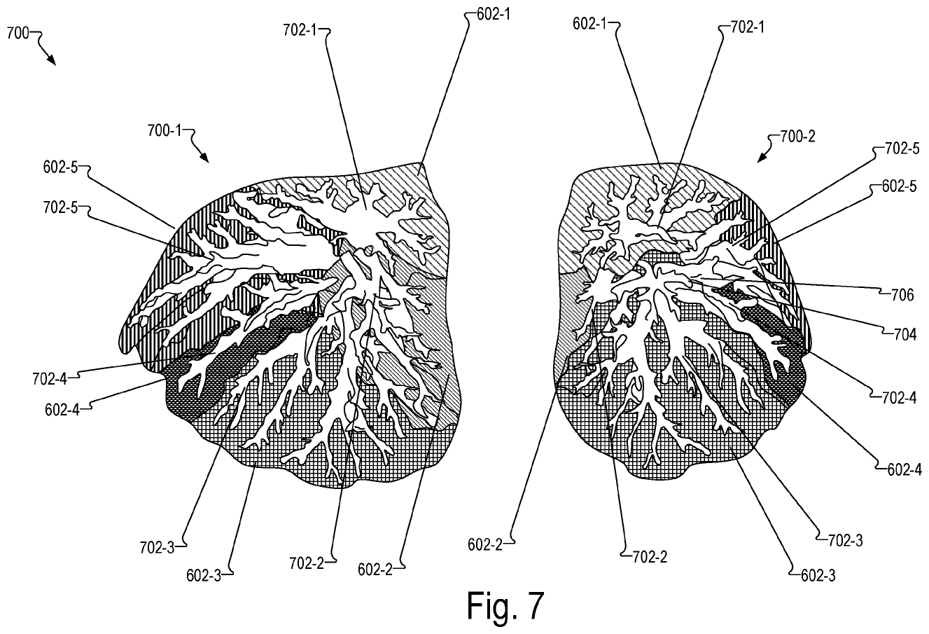

FIG. 7 illustrates additional example images 700 (e.g., images 700-1 and 700-2) of segments 602 determined based on configurations 400 and 500. Images 700 show segments 602 with their respective seeds 702 (e.g., seeds 702-1 through 702-5). As described, each seed 702 includes a respective labeled airway vessel branch 406 and a correspondingly labeled artery vessel branch 410. Based on the set of seeds, system 100 may determine a partitioning that defines boundaries of each segment 602.

In some examples, by including entire labeled airway vessel branches 406 and artery vessel branches 410 (as opposed to structures derived from one or both) as seeds 702, the resulting segments 602 determined based on seeds 702 may be ensured to contain a respective seed 702. For example, boundaries of segments 602 determined based on such seeds 702 may be ensured to not cross the labeled vessel structures 204, as would be expected from actual boundaries of functional segments of organs. Further, including artery vessel structure 408 in seeds 702 may result in segments 602 that contain the artery vessels (e.g., boundaries of segments do not cross arteries), which may result in more accurate segment partitioning than if seeds 702 were based on airway vessel structure 404 alone. By basing seeds 702 on airway vessel structure 404 and artery vessel structure 408, each branch of the labeled vessel structures may be contained within a respective set of the boundaries of the segments. As one example, image 700-2 shows a labeled vessel branch portion 704 contained within a boundary 706 of segments. Specifically, the labeled vessel branch portion 704 is contained entirely within segment 602-3.

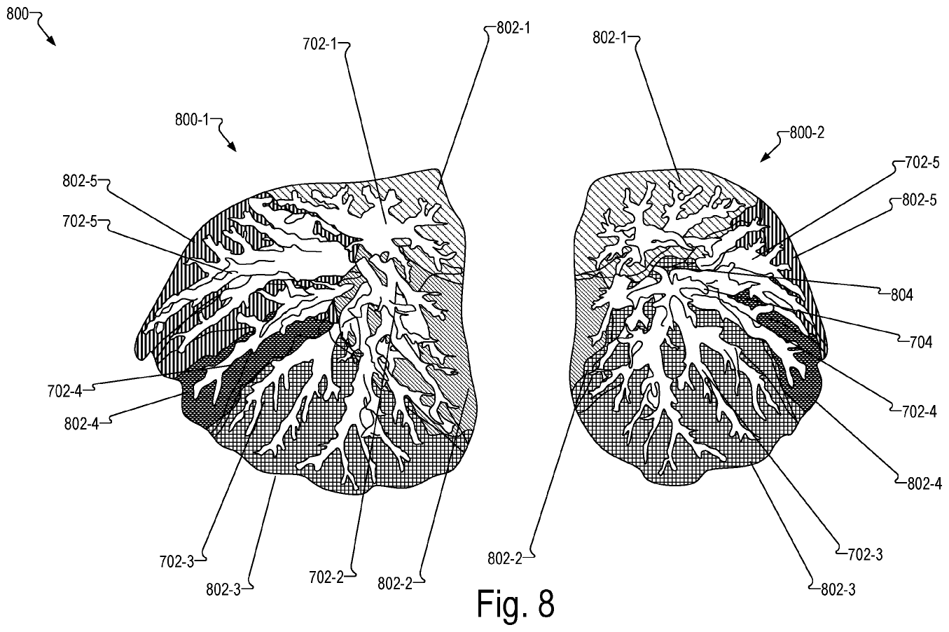

For instance, FIG. 8 illustrates example images 800 (e.g., images 800-1 and 800-2) that show a partitioning of organ model 202 based on airway vessel structure 404 alone (e.g., seeds do not include artery vessel structure 408). Resulting segments 802 (e.g., segments 802-1 through 802-5) may be different from segments 602 as determined based on seeds 702.

For example, images 800 show segments 802 and seeds 702 (seeds as shown in images 700, including airway vessel branches 406 and artery vessel branches 410). As shown, segments 802 may not contain (e.g., fully enclose) seeds 702, specifically artery vessel branches 410. Rather, as one example, labeled vessel branch portion 704 (which was shown as contained within boundary 706 in FIG. 7) is shown in image 800-2 with a boundary 804 of segments that is different from boundary 706 and crosses labeled vessel branch portion 704. Such a boundary would likely not accurately reflect an actual boundary of a bronchopulmonary segment.

In some examples, based on the partitioning of organ model 202 into segments 602, system 100 may determine additional information and output such information to a user of system 100. For instance, system 100 may be configured to determine, based on the partitioning, a volume of one or more of segments 602. Based on the volume, system 100 may determine an estimation of function loss based on a removal of one or more of segments 602 and/or a portion of one or more of segments 602. As another example, system 100 may provide an output that alerts a user of a computer-assisted medical system (e.g., a surgeon), that the surgeon is approaching a boundary of a segment 602 during a medical procedure. Any other such suitable information based on the partitioning may be determined and output by system 100 and/or any other suitable system.

While examples herein have shown a partitioning of a lung, systems and methods may be applied to any suitable organ using any suitable vessel structure and/or vessel structures. For instance, system 100 may be configured to partition a liver based on a portal vein vessel structure and a hepatic vein vessel structure.

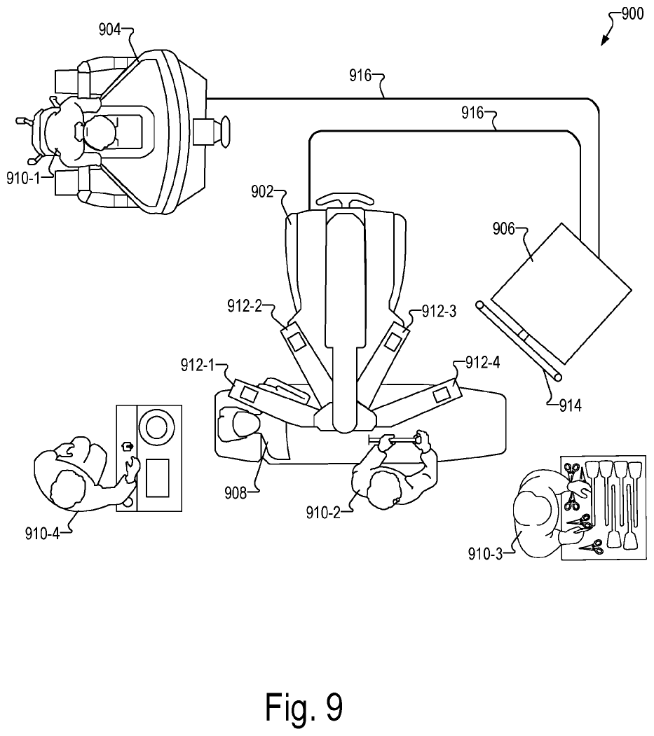

As has been described, system 100 may be associated in certain examples with a computer-assisted medical system used to perform a medical procedure on a subject. To illustrate, FIG. 9 shows an illustrative computer-assisted medical system 900 that may be used to perform various types of medical procedures including surgical and/or non-surgical procedures.

As shown, computer-assisted medical system 900 may include a manipulator assembly 902 (a manipulator cart is shown in FIG. 9), a user control apparatus 904, and an auxiliary apparatus 906, all of which are communicatively coupled to each other. Computer-assisted medical system 900 may be utilized by a medical team to perform a computer-assisted medical procedure or other similar operation on a body of a subject 908 or on any other body as may serve a particular implementation. As shown, the medical team may include a first user 910-1 (such as a surgeon for a surgical procedure), a second user 910-2 (such as a subject-side assistant), a third user 910-3 (such as another assistant, a nurse, a trainee, etc.), and a fourth user 910-4 (such as an anesthesiologist for a surgical procedure), all of whom may be collectively referred to as users 910, and each of whom may control, interact with, or otherwise be a user of computer-assisted medical system 900. More, fewer, or alternative users may be present during a medical procedure as may serve a particular implementation. For example, team composition for different medical procedures, or for non-medical procedures, may differ and include users with different roles.

While FIG. 9 illustrates an ongoing minimally invasive medical procedure such as a minimally invasive surgical procedure, it will be understood that computer-assisted medical system 900 may similarly be used to perform open medical procedures or other types of operations. For example, operations such as exploratory imaging operations, mock medical procedures used for training purposes, and/or other operations may also be performed.

As shown in FIG. 9, manipulator assembly 902 may include one or more manipulator arms 912 (e.g., manipulator arms 912-1 through 912-4) to which one or more instruments may be coupled. The instruments may be used for a computer-assisted medical procedure on subject 908 (e.g., in a surgical example, by being at least partially inserted into subject 908 and manipulated within subject 908). While manipulator assembly 902 is depicted and described herein as including four manipulator arms 912, it will be recognized that manipulator assembly 902 may include a single manipulator arm 912 or any other number of manipulator arms as may serve a particular implementation. While the example of FIG. 9 illustrates manipulator arms 912 as being robotic manipulator arms, it will be understood that, in some examples, one or more instruments may be partially or entirely manually controlled, such as by being handheld and controlled manually by a person. For instance, these partially or entirely manually controlled instruments may be used in conjunction with, or as an alternative to, computer-assisted instrumentation that is coupled to manipulator arms 912 shown in FIG. 9.

During the medical operation, user control apparatus 904 may be configured to facilitate teleoperational control by user 910-1 of manipulator arms 912 and instruments attached to manipulator arms 912. To this end, user control apparatus 904 may provide user 910-1 with imagery of an operational area associated with subject 908 as captured by an imaging device. To facilitate control of instruments, user control apparatus 904 may include a set of master controls. These master controls may be manipulated by user 910-1 to control movement of the manipulator arms 912 or any instruments coupled to manipulator arms 912.

Auxiliary apparatus 906 may include one or more computing devices configured to perform auxiliary functions in support of the medical procedure, such as providing insufflation, electrocautery energy, illumination or other energy for imaging devices, image processing, or coordinating components of computer-assisted medical system 900. In some examples, auxiliary apparatus 906 may be configured with a display monitor 914 configured to display one or more user interfaces, or graphical or textual information in support of the medical procedure. In some instances, display monitor 914 may be implemented by a touchscreen display and provide user input functionality. Augmented content provided by a region-based augmentation system may be similar, or differ from, content associated with display monitor 914 or one or more display devices in the operation area (not shown).

Manipulator assembly 902, user control apparatus 904, and auxiliary apparatus 906 may be communicatively coupled one to another in any suitable manner. For example, as shown in FIG. 9, manipulator assembly 902, user control apparatus 904, and auxiliary apparatus 906 may be communicatively coupled by way of control lines 916, which may represent any wired or wireless communication link as may serve a particular implementation. To this end, manipulator assembly 902, user control apparatus 904, and auxiliary apparatus 906 may each include one or more wired or wireless communication interfaces, such as one or more local area network interfaces, Wi-Fi network interfaces, cellular interfaces, and so forth.

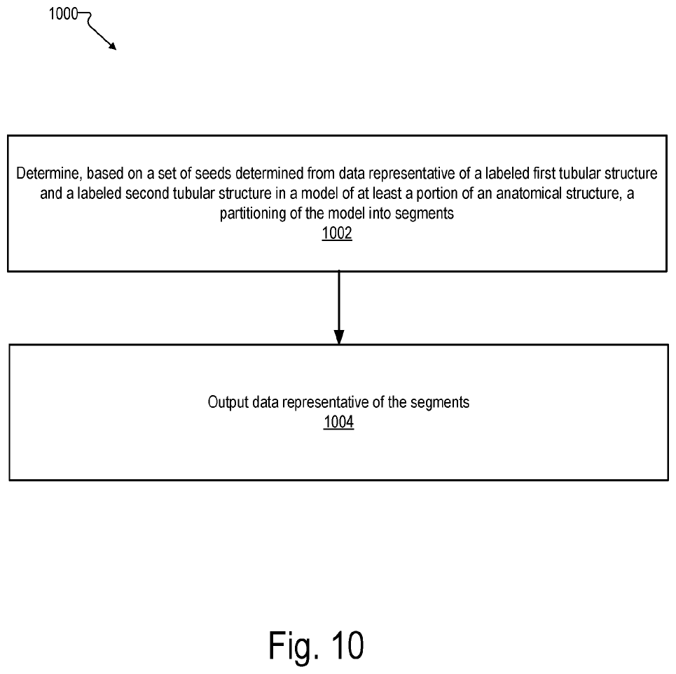

FIG. 10 illustrates an example method 1000 of an anatomical structure model partitioning system. While FIG. 10 illustrates example operations according to one embodiment, other embodiments may omit, add to, reorder, combine, and/or modify any of the operations shown in FIG. 10. One or more of the operations shown in in FIG. 10 may be performed by an anatomical structure model partitioning system such as system 100, any components included therein, and/or any implementation thereof.

At operation 1002, an anatomical structure model partitioning system may determine, based on a set of seeds determined from data representative of a labeled first tubular structure and a labeled second tubular structure in a model of at least a portion of an anatomical structure, a partitioning of the model into segments. Operation 1002 may be performed in any of the ways described herein.

At operation 1004, the anatomical structure model partitioning system may output data representative of the segments. Operation 1004 may be performed in any of the ways described herein.

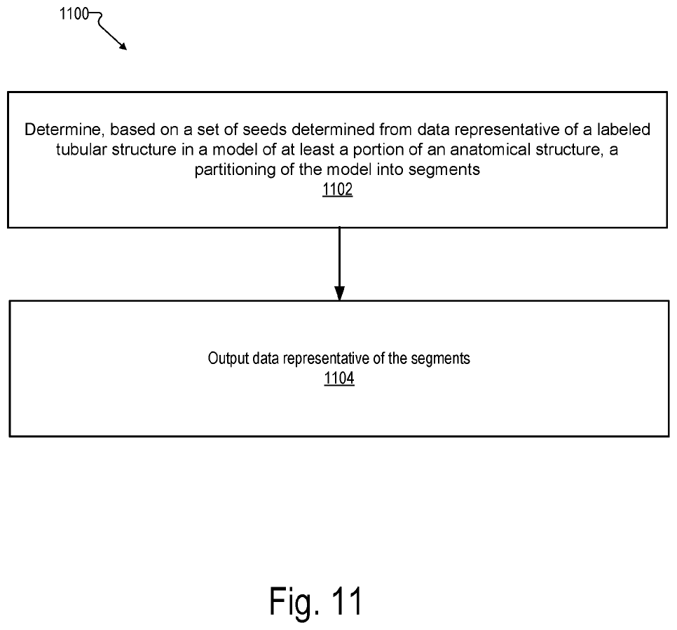

FIG. 11 illustrates an example method 1100 of an anatomical structure model partitioning system. While FIG. 11 illustrates example operations according to one embodiment, other embodiments may omit, add to, reorder, combine, and/or modify any of the operations shown in FIG. 11. One or more of the operations shown in in FIG. 11 may be performed by an anatomical structure model partitioning system such as system 100, any components included therein, and/or any implementation thereof.

At operation 1102, an anatomical structure model partitioning system may determine, based on a set of seeds determined from data representative of a labeled tubular structure in a model of at least a portion of an anatomical structure, a partitioning of the model into segments. Operation 1102 may be performed in any of the ways described herein.

At operation 1104, the anatomical structure model partitioning system may output data representative of the segments. Operation 1104 may be performed in any of the ways described herein.

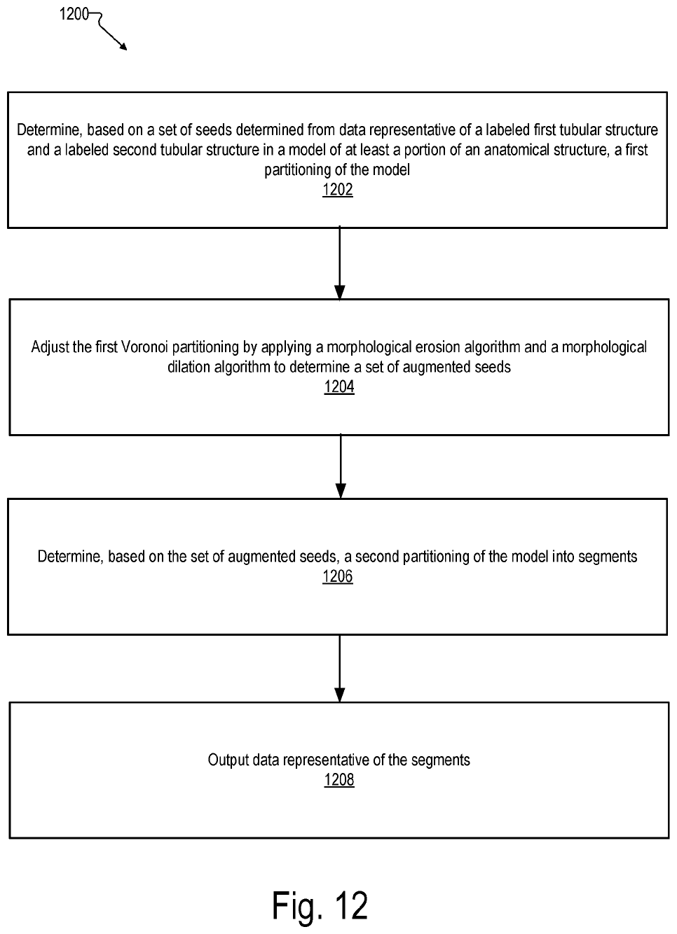

FIG. 12 illustrates an example method 1200 of an anatomical structure model partitioning system. While FIG. 12 illustrates example operations according to one embodiment, other embodiments may omit, add to, reorder, combine, and/or modify any of the operations shown in FIG. 12. One or more of the operations shown in in FIG. 12 may be performed by an anatomical structure model partitioning system such as system 100, any components included therein, and/or any implementation thereof.

At operation 1202, an anatomical structure model partitioning system may determine, based on a set of seeds determined from data representative of a labeled first tubular structure and a labeled second tubular structure in a model of at least a portion of an anatomical structure, a first partitioning of the model (e.g., a first Voronoi partitioning of the model). Operation 1202 may be performed in any of the ways described herein.

At operation 1204, the anatomical structure model partitioning system may adjust the first Voronoi partitioning by applying a morphological erosion algorithm and a morphological dilation algorithm to determine a set of augmented seeds. Operation 1204 may be performed in any of the ways described herein.

At operation 1206, the anatomical structure model partitioning system may determine, based on the set of augmented seeds, a second partitioning of the model (e.g., a second Voronoi partitioning of the model) into segments. Operation 1206 may be performed in any of the ways described herein.

At operation 1208, the anatomical structure model partitioning system may output data representative of the segments. Operation 1208 may be performed in any of the ways described herein.

In some examples, a non-transitory computer-readable medium storing computer-readable instructions may be provided in accordance with the principles described herein. The instructions, when executed by a processor of a computing device, may direct the processor and/or computing device to perform one or more operations, including one or more of the operations described herein. Such instructions may be stored and/or transmitted using any of a variety of known computer-readable media.

A non-transitory computer-readable medium as referred to herein may include any non-transitory storage medium that participates in providing data (e.g., instructions) that may be read and/or executed by a computing device (e.g., by a processor of a computing device). For example, a non-transitory computer-readable medium may include, but is not limited to, any combination of non-volatile storage media and/or volatile storage media. Illustrative non-volatile storage media include, but are not limited to, read-only memory, flash memory, a solid-state drive, a magnetic storage device (e.g. a hard disk, a floppy disk, magnetic tape, etc.), ferroelectric random-access memory (“RAM”), and an optical disc (e.g., a compact disc, a digital video disc, a Blu-ray disc, etc.). Illustrative volatile storage media include, but are not limited to, RAM (e.g., dynamic RAM).

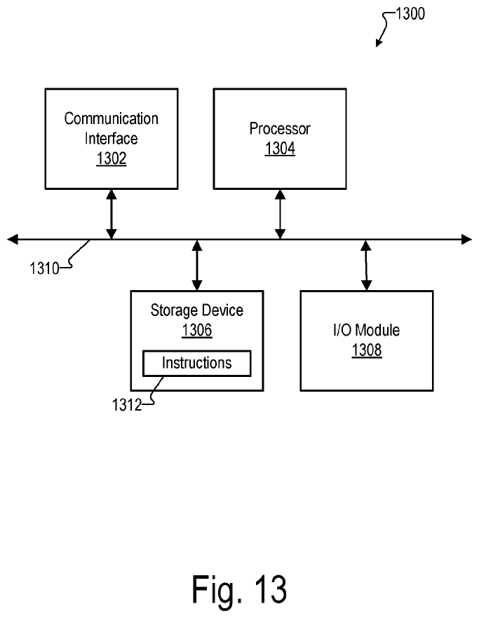

FIG. 13 illustrates an example computing device 1300 that may be specifically configured to perform one or more of the processes described herein. Any of the systems, units, computing devices, and/or other components described herein may implement or be implemented by computing device 1300.

As shown in FIG. 13, computing device 1300 may include a communication interface 1302, a processor 1304, a storage device 1306, and an input/output (“I/O”) module 1308 communicatively connected one to another via a communication infrastructure 1310. While an example computing device 1300 is shown in FIG. 13, the components illustrated in FIG. 13 are not intended to be limiting. Additional or alternative components may be used in other embodiments. Components of computing device 1300 shown in FIG. 13 will now be described in additional detail.

Communication interface 1302 may be configured to communicate with one or more computing devices. Examples of communication interface 1302 include, without limitation, a wired network interface (such as a network interface card), a wireless network interface (such as a wireless network interface card), a modem, an audio/video connection, and any other suitable interface.

Processor 1304 generally represents any type or form of processing unit capable of processing data and/or interpreting, executing, and/or directing execution of one or more of the instructions, processes, and/or operations described herein. Processor 1304 may perform operations by executing computer-executable instructions 1312 (e.g., an application, software, code, and/or other executable data instance) stored in storage device 1306.

Storage device 1306 may include one or more data storage media, devices, or configurations and may employ any type, form, and combination of data storage media and/or device. For example, storage device 1306 may include, but is not limited to, any combination of the non-volatile media and/or volatile media described herein. Electronic data, including data described herein, may be temporarily and/or permanently stored in storage device 1306. For example, data representative of computer-executable instructions 1312 configured to direct processor 1304 to perform any of the operations described herein may be stored within storage device 1306. In some examples, data may be arranged in one or more databases residing within storage device 1306.

I/O module 1308 may include one or more I/O modules configured to receive user input and provide user output. 1/O module 1308 may include any hardware, firmware, software, or combination thereof supportive of input and output capabilities. For example, I/O module 1308 may include hardware and/or software for capturing user input, including, but not limited to, a keyboard or keypad, a touchscreen component (e.g., touchscreen display), a receiver (e.g., an RF or infrared receiver), motion sensors, and/or one or more input buttons.

I/O module 1308 may include one or more devices for presenting output to a user, including, but not limited to, a graphics engine, a display (e.g., a display screen), one or more output drivers (e.g., display drivers), one or more audio speakers, and one or more audio drivers. In certain embodiments, I/O module 1308 is configured to provide graphical data to a display for presentation to a user. The graphical data may be representative of one or more graphical user interfaces and/or any other graphical content as may serve a particular implementation.

In some examples, any of the systems, modules, and/or facilities described herein may be implemented by or within one or more components of computing device 1300. For example, one or more applications 1312 residing within storage device 1306 may be configured to direct an implementation of processor 1304 to perform one or more operations or functions associated with processing facility 104 of system 100.

As mentioned, one or more operations described herein may be performed during a medical procedure, e.g., dynamically, in real time, and/or in near real time. As used herein, operations that are described as occurring “in real time” will be understood to be performed immediately and without undue delay, even if it is not possible for there to be absolutely zero delay.

Any of the systems, devices, and/or components thereof may be implemented in any suitable combination or sub-combination. For example, any of the systems, devices, and/or components thereof may be implemented as an apparatus configured to perform one or more of the operations described herein.

In the description herein, various example embodiments have been described. It will, however, be evident that various modifications and changes may be made thereto, and additional embodiments may be implemented, without departing from the scope of the invention as set forth in the claims that follow. For example, certain features of one embodiment described herein may be combined with or substituted for features of another embodiment described herein. The description and drawings are accordingly to be regarded in an illustrative rather than a restrictive sense.

1-38. (canceled)

39. A system comprising:
a memory storing instructions; and
a processor communicatively coupled to the memory and configured to execute the instructions to perform a process comprising:
determining, based on a set of seeds determined from data representative of a labeled first tubular structure comprising a blood vessel structure and a labeled second tubular structure comprising an airway vessel structure in a model of at least a portion of an anatomical structure comprising a lung, a partitioning of the model into segments comprising bronchopulmonary segments; and
outputting data representative of the segments.

40. The system of claim 39, wherein the determining the partitioning comprises:
determining a first partitioning based on the set of seeds;
adjusting the first partitioning to determine a set of augmented seeds; and
determining, based on the set of augmented seeds, a second partitioning of the model into the segments.

41. The system of claim 40, wherein the adjusting the first partitioning comprises applying at least one of a smoothing algorithm, a denoising algorithm, or a tolerance algorithm.

42. The system of claim 40, wherein the adjusting the first partitioning comprises applying at least one of a dilation algorithm or an expansion algorithm.

43. The system of claim 40, wherein the adjusting the first partitioning comprises applying at least one of a morphological erosion algorithm or a morphological dilation algorithm.

44. The system of claim 40, wherein at least one of the determining the first partitioning or the determining the second partitioning comprises applying a nearest neighbor algorithm.

45. The system of claim 40, wherein at least one of the determining the first partitioning or the determining the second partitioning comprises determining a Voronoi partitioning.

46. The system of claim 39, wherein:
the partitioning defines boundaries of the segments; and
each labeled tubular branch of the labeled first tubular structure is contained within a respective set of the boundaries of the segments.

47. The system of claim 39, further comprising:
determining a volume of one or more of the segments; and
determining, based on the volume, an estimation of function loss based on a removal of one or more of the segments.

48. A method comprising:
determining, by a computing system and based on a set of seeds determined from data representative of a labeled tubular structure comprising a blood vessel structure in a model of at least a portion of an anatomical structure comprising a lung, a partitioning of the model into segments comprising bronchopulmonary segments; and
outputting, by the computing system, data representative of the segments.

49. The method of claim 48, wherein the set of seeds is determined further based on data representative of an additional labeled tubular structure in the model.

50. The method of claim 48, wherein the determining the partitioning comprises:
determining a first partitioning based on the set of seeds;
adjusting the first partitioning to determine a set of augmented seeds; and
determining, based on the set of augmented seeds, a second partitioning of the model into the segments.

51. The method of claim 50, wherein the adjusting the first partitioning comprises applying at least one of a smoothing algorithm, a denoising algorithm, or a tolerance algorithm.

52. The method of claim 50, wherein the adjusting the first partitioning comprises applying at least one of a dilation algorithm or an expansion algorithm.

53. The method of claim 50, wherein the adjusting the first partitioning comprises applying at least one of a morphological erosion algorithm or a morphological dilation algorithm.

54. The method of claim 50, wherein at least one of the determining the first partitioning or the determining the second partitioning comprises applying a nearest neighbor algorithm.

55. The method of claim 50, wherein at least one of the determining the first partitioning or the determining the second partitioning comprises determining a Voronoi partitioning.

56. The method of claim 48, wherein:
the partitioning defines boundaries of the segments; and
each labeled tubular branch of the labeled tubular structure is contained within a respective set of the boundaries of the segments.

57. The method of claim 48, further comprising:
determining, by the computing system, a volume of one or more of the segments; and
determining, by the computing system and based on the volume, an estimation of function loss based on a removal of one or more of the segments.

58. A non-transitory computer-readable medium storing instructions that, when executed, direct a processor of a computing device to perform a process comprising:
determining, based on a set of seeds determined from data representative of a labeled tubular structure comprising a blood vessel structure and an additional labeled tubular structure comprising an airway vessel structure in a model of at least a portion of an anatomical structure comprising a lung, a partitioning of the model into segments comprising bronchopulmonary segments; and
outputting, data representative of the segments.
